(19) United States
(12) Patent Application Publication
(10) Pub. No.: US 2007/0077450 A1
(43) Pub. Date: Apr. 5, 2007

(54) Conjugated compounds containing triarylamine structural elements, and their use

(76) Inventors: Feng-wen Yen, Hsinchu City (TW); Chao-Yu Chiu, Hsinchu City (TW); Cheng-Hao Chang, Miaoli County (TW); Chin-Ming Teng, Miaoli County (TW); Feng-Ying Wang, Taipei (TW); Yung-Fan Cheng, Miaoli County (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/242,007

(22) Filed: Oct. 4, 2005

(51) Int. Cl. H01L 51/54; C07C 211/60

(52) U.S. Cl. 428/690; 428/917; 313/504; 313/506; 257/40; 257/E51.051; 564/426; 564/434

(57) ABSTRACT

The present invention discloses conjugated compounds containing triarylamine structural elements with a general formula ABₘ, wherein A is aryl moiety with or without hetero atom(s), m is integers from 2 to 4, and B is as following: 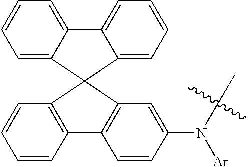 wherein Ar is aryl moiety. Moreover, the conjugated compounds of the invention are used as hole transport material, hole injection material and/or as host material in organic electroluminescence devices and/or phosphorescence devices, and as hole transport material in solar cells.

### BACKGROUND OF THE INVENTION

1. Field of the Invention

The present invention is generally related to conjugated compounds, and more particularly to conjugated compounds containing triarylamine structural elements and their use.

2. Description of the Prior Art

The main structural unit of interest is the triarylamine (TAA) moiety which is used as hole transport material (hereinafter HTMs) of solar cells. The synthetic programme was extensively based on the palladium-mediated coupling reactions developed by Buchwald and Hartwig. However, for lack of good thermal stability and dimensional stability, intensive research has been focused on the development of new hole-transporting materials.

The radically different manufacturing process of organic light emitting diodes (OLEDs) lends itself to many advantages over traditional flat panel displays. Since OLEDs can be printed onto a substrate using traditional inkjet technology they can have a significantly lower cost than LCDs or plasma displays. A more scalable manufacturing process enables the possibility of much larger displays. Unlike LCDs which employ a back-light and are incapable of showing true black, an off OLED element produces no light allowing for infinite contrast ratios. The range of colors, brightness, and viewing angle possible with OLEDs is greater than that of LCDs or plasma displays. Without the need of a backlight, OLEDs use less than half the power of LCD displays and are well-suited to mobile applications such as cell phones and digital cameras.

The working principle of OLEDs is that the holes and the electrons which are injected from the anode and cathodes respectively. The holes will inject into the Highest Occupied Molecular Orbital (HOMO) of the hole transporting layer, and electrons will inject into the Lowest Unoccupied Molecular Orbital (LUMO) of the electron transporting layer. With the voltage difference across the OLED, the holes and electrons are able to migrate in the organic layers. Finally, electrons and holes recombine on the same polymer chain or molecule to form electron-hole pairs in the emitting layer, so that light emission can occur.

The most frequently used hole transporting materials, such as N,N′-di-m-tolyl-N,N′-diphenyl-1,1′-biphenyl-4,4′-diamine (TPD) and 4,4′-bis[N-(1-naphthyl)-N-phenylamino]biphenyl (NPB) have been proved to be excellent hole-transporting materials and have shown a wide range of practical applications. These classes of materials offer many attractive properties such as high charge carrier mobility and ease of sublimation. However, they possess some disadvantages for use in long-lifetime OLED devices such as their relatively low glass transition temperature (Tg ˜65° C. for TPD and Tg ˜100° C. for NPB), ease of crystallization and unsatisfactory morphological stability. Therefore, new hole transporting materials are still needed corresponding to increasing thermal stability and decreasing injection barrier between hole transporting layer and anode, so as to improve the efficiency and to extend the lifetime of OLEDs.

### SUMMARY OF THE INVENTION

In accordance with the present invention, new conjugated compounds containing triarylamine structural elements and their use are provided. These new conjugated compounds can overcome the drawbacks of the mentioned conventional skill.

One object of the present invention is to have spiroarylamines reacted with multihalo-aryl compounds and a base in the presence of a palladium component, so as to form the mentioned conjugated compounds containing triarylamine structural elements.

Another object of the present invention is to provide a multilayered OLED with high brightness, high efficiency, and high thermal stability, wherein the OLED comprises the mentioned conjugated compounds containing triarylamine structural elements as hole transporting material or hole injecting material. The glass transition temperature (T_{g}) and thermal degradation temperature (T_{d}) of the mentioned conjugated compounds are 279° C. and 532° C. respectively, better than that of NPB. Therefore, this present invention does have the economic advantages for industrial applications.

Accordingly, the present invention discloses conjugated compounds containing triarylamine structural elements with a general formula ABₘ, wherein A is aryl moiety with or without hetero atom(s), m is integers from 2 to 4, and B is as following: 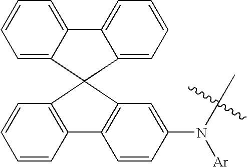
wherein Ar is aryl moiety. Moreover, the conjugated compounds of the invention are used as hole transport material, hole injection material and/or as host material in organic electroluminescence devices and/or phosphorescence devices, and as hole transport material in solar cells.

### BRIEF DESCRIPTION OF THE DRAWINGS

FIG. 1A shows plots of luminance v. voltage for devices I-a, I-b, I-c, and I-d;

FIG. 1B shows plots of current density v. voltage for devices I-a, I-b, I-c, and I-d;

FIG. 1C shows plots of EL efficiency v. current density for devices I-a, I-b, I-c, and I-d;

FIG. 2A shows plots of luminance v. voltage for devices II-a, II-b, II-c, and II-d;

FIG. 2B shows plots of current density v. voltage for devices II-a, II-b, II-c, and II-d; and

FIG. 2C shows plots of EL efficiency v. current density for devices II-a, II-b, II-c, and II-d.

### DESCRIPTION OF THE PREFERRED EMBODIMENTS

What probed into the invention are conjugated compounds containing triarylamine structural elements and their use. Detailed descriptions of the production, structure and elements will be provided in the following in order to make the invention thoroughly understood. Obviously, the application of the invention is not confined to specific details familiar to those who are skilled in the art. On the other hand, the common elements and procedures that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

In a first embodiment of the present invention, a conjugated compound containing triarylamine structural elements with a general formula ABₘ is provided, wherein A is aryl moiety with or without hetero atom(s), m is integers from 2 to 4, and B is as following: 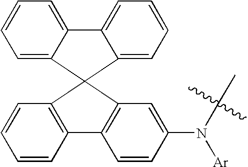
wherein Ar is aryl moiety and selected from the group consisting of:

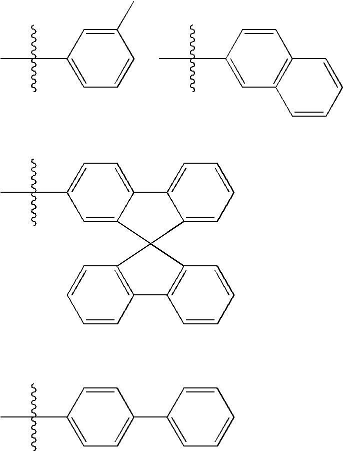

In this embodiment, A is selected from the group consisting of: 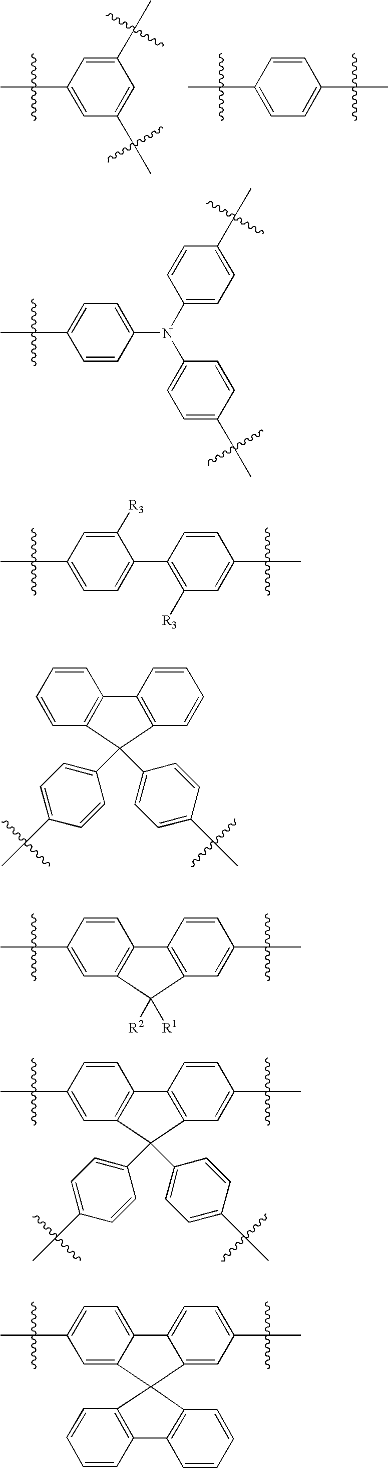 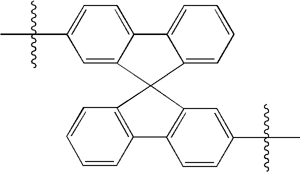
R¹ and R² are identical or different, and R¹ and R² are independently selected from the group consisting of: linear alkyl, branched alkyl, cyclic alkyl, aryl moiety, hetero cycle, multiple fused ring, multiple fused ring with hetero atom(s), and alkyl with at least one substitute of alkene or alkyne or halogen or alkoxy or siloxane or ketone or alcohol or thioether or carbamates, or amine. In a preferred example of this embodiment, R¹ and R² are identical or different, and both of R¹ and R² are aryl moiety. Additionally, R³ is selected from the group consisting of: hydrogen, halogen, linear alkyl, branched alkyl, cyclic alkyl, aryl moiety, hetero cycle, multiple fused ring, multiple fused ring with hetero atom(s), and alkyl with at least one substitute of alkene or alkyne or halogen or alkoxy or siloxane or ketone or alcohol or thioether or carbamates, or amine.

### EXAMPLE 1

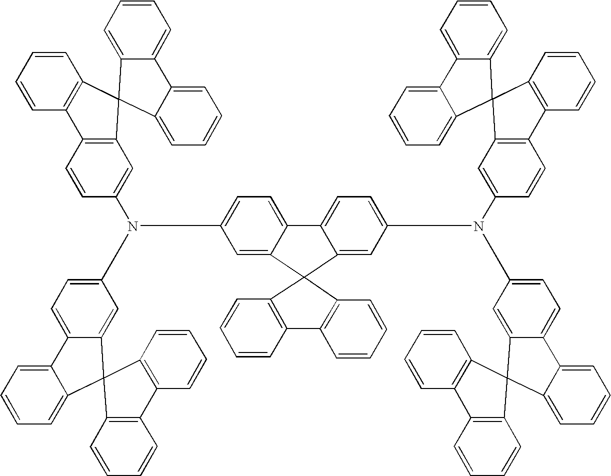

IR (KBr) □ 3065, 3010, 2955, 2918, 2869, 1946, 1916, 1794, 1708, 1604, 1568, 1494, 1421, 1348, 1317, 1219, 1152 cm⁻¹; ¹H NMR (C₂D₂Cl₄, 400 MHz): 7.70 (d, J=7.6 Hz, 16H), 7.54 (d, J=7.6 Hz, 4H), 7.28 (t, J=7.2 Hz, 16H), 7.15-7.24 (m, 4H), 7.02 (t, J=7.6 Hz, 12H), 6.96 (t, J=8.0 Hz, 2H), 6.56 (d, J=7.2 Hz, 16H), 6.45 (d, J=5.6 Hz, 4H); ¹³C NMR (CDCl₃, 100 MHz): 147.98, 140.94, 140.67, 128.62, 127.81, 127.35, 127.09, 126.94, 124.91, 123.42, 123.23, 119.44, 119.22, 65.87; MS (m/z, FAB⁺) 1603.4 (100%).

### EXAMPLE 2

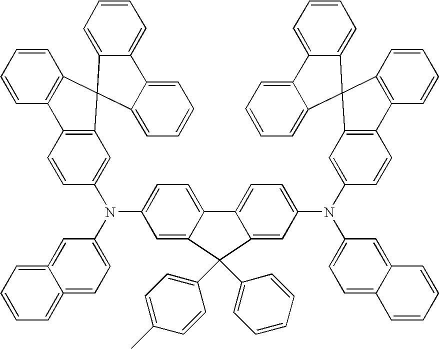

IR (KBr) □ 3053, 2907, 1904, 1933, 1839, 1810, 1740, 1728, 1646, 1605, 1558, 1499, 1394, 1347, 1311, 1211 cm⁻¹; ¹H NMR (acetone-d₆, 400 MHz): 7.83 (d, J=2.8 Hz, 2H), 7.81 (d, J=4.0 Hz, 2H), 7.74-7.78 (m, 6H), 7.66 (d, J=8.4 Hz, 2H), 7.62 (d, J=8.4 Hz, 2H), 7.40-7.42 (m, 4H), 7.31 (t, J=7.2 Hz, 4H), 7.26 (dt, J=7.6, 1.2 Hz, 4H), 7.19 (dt, J=7.2, 1.2 Hz, 2H), 7.07-7.10 (m, 6H), 7.01 (dt, J=8.0, 1.2 Hz, 4H), 6.95 (dd, J=8.0, 2.0 Hz, 2H), 6.87 (dd, J=8.0, 2.4 Hz, 4H), 6.66-6.70 (m, 7H), 6.59 (d, J=7.2 Hz, 2H), 6.51 (d, J=7.2 Hz, 2H), 6.46 (d, J=8.0 Hz, 2H), 6.37 (d, J=2.0 Hz, 2H), 2.17 (s, 3H); ¹³C NMR (acetone-d₆, 100 MHz): 152.15, 150.07, 148.84, 148.66, 148.38, 147.20, 145.50, 143.49, 142.43, 141.58, 136.20, 135.87, 135.48, 133.97, 130.43, 128.82, 128.65, 127.98, 127.76, 127.69, 127.06, 126.52, 126.31, 124.16, 123.76, 123.62, 122.23, 121.20, 120.42, 119.81, 117.55, 66.54, 65.25, 21.06; MS (m/z, FAB⁺) 1242.5 (100%).

### EXAMPLE 3

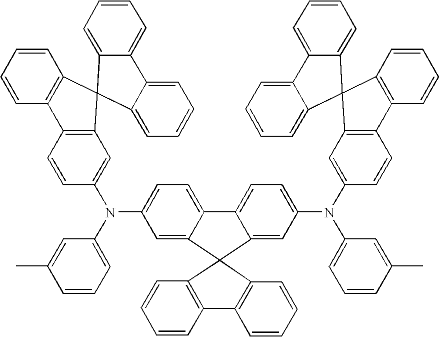

IR (KBr) □ 3050, 2999, 2292, 1719, 1599, 1567, 1497, 1446, 1344, 1306, 1268, 1210 cm⁻¹; ¹H NMR (acetone-d₆, 400 MHz): 7.84 (dd, J=7.4, 2.4 Hz, 2H), 7.75 (d, J=8.4 Hz, 2H), 7.70 (d, J=8.0 Hz, 2H), 7.64 (d, J=8.4 Hz, 2H), 7.32 (dt, J=7.2, 1.6 Hz, 6H), 7.26 (dt, J=7.4, 1.2 Hz, 2H), 7.11 (dt, J=7.2, 0.8 Hz, 6H), 7.04 (dt, J=7.4, 1.2 Hz, 2H), 6.90 (dt, J=7.8, 2.4 Hz, 4H), 6.76 (t, J=8.0 Hz, 2H), 6.70 (d, J=7.2 Hz, 2H), 6.66 (d, J=7.2 Hz, 2H), 6.56 (d, J=8.0 Hz, 2H), 6.53 (d, J=7.2 Hz, 2H), 6.49 (s, 2H), 6.43 (d, J=8.4 Hz, 2H), 6.31 (d, J=2.0 Hz, 2H), 6.25 (d, J=1.6 Hz, 2H), 1.90 (s, 6H); ¹³C NMR (acetone-d₆, 100 MHz): 150.18, 150.14, 148.69, 148.66, 148.46, 147.30, 146.67, 141.70, 141.56, 141.50, 138.72, 136.79, 136.65, 128.93, 128.06, 128.03, 128.00, 127.26, 124.03, 123.77, 123.73, 123.65, 121.17, 120.68, 120.44, 120.41, 119.94, 119.67, 119.49, 66.47, 66.34, 21.26; MS (m/z, FAB⁺) 1154.5 (100%).

### EXAMPLE 4

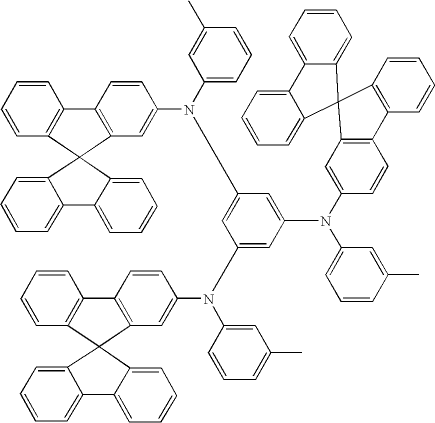

IR(CH₂Cl₂) ν 3945, 3441, 3054, 2987, 2305, 1633, 1422, 1265, 896, 739 cm⁻¹; ¹H NMR (Acetone-d₆, 400 MHz) δ 7.89 (d, J=8.0 Hz, 6H), 7.82 (d, J=8.0 Hz, 9H), 7.37 (t, J=7.4 Hz, 3H), 7.20 (t, J=7.4 Hz, 6H), 7.08 (t, J=7.60 Hz, 3H), 6.95-6.86 (m, 12H), 6.57 (t, J=7.0 Hz, 9H), 6.50-6.45 (m, 9H), 6.30 (s, 3H), 6.02 (s, 3H), 2.02 (s, 9H); ¹³C NMR (Acetone-d6, 100 MHz) δ 204.9, 150.1, 148.9, 148.7, 148.6, 147.1, 146.8, 141.7, 138.6, 137.0, 128.9, 128.0, 127.3, 124.8, 123.8, 123.7, 123.4, 121.2, 120.5, 120.3, 120.2, 120.0, 113.7, 104.6, 66.4, 21.5,; MS (m/z, FAB⁺) 1336.3

### EXAMPLE 5

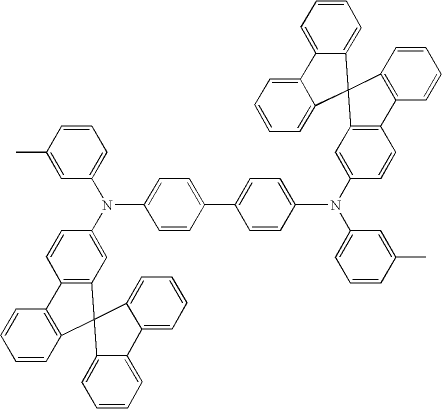

IR(CH₂Cl₂) ν 3944, 3686, 3442, 3054, 2987, 2306, 1422, 1265, 896, 737, 705 cm⁻¹; ¹H NMR (CDCl₃, 400 MHz) δ 7.77 (t, J=7.0 Hz, 6H), 7.70 (d, J=8.4 Hz, 2H), 7.34 (t, J=7.6 Hz, 6H), 7.29 (d, J=8.4 Hz, 4H), 7.14 (t, J=8.0 Hz, 4H), 7.07-6.98 (m, 10H), 6.84 (d, J=7.2 Hz, 4H), 6.80-6.69 (m, 8H), 6.61 (s, 2H), 2.15 (s, 6H); ¹³C NMR (CDCl₃, 100 MHz) δ 149.8, 148.6, 148.5, 147.2, 147.1, 146.2, 141.5, 141.3, 138.7, 138.5, 134.4, 128.7, 127.6, 127.5, 127.0, 126.9, 124.2, 124.0, 123.8, 123.4, 120.9, 120.5, 120.0, 119.8, 119.3, 66.3, 22.0; MS (m/z, FAB⁺) 992.3

### EXAMPLE 6

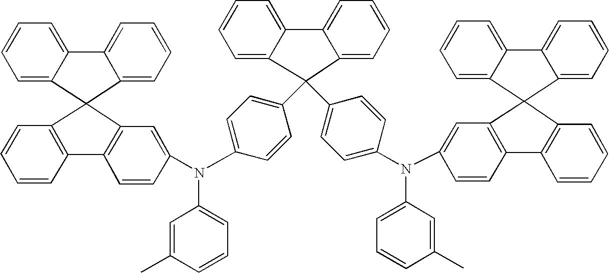

IR(CH₂Cl₂) ν 3944, 3686, 3442, 3054, 2987, 2306, 1422, 1265, 896, 737, 705 cm⁻¹; ¹H NMR (CDCl₃, 400 MHz) δ 7.73˜7.70 (m, 10H), 7.67 (d, J=8.4 Hz, 2H), 7.37˜7.24 (m, 10H), 7.08˜6.94 (m, 10H), 6.84 (d, J=8.4 Hz, 4H), 6.78˜6.72 (m, 10H), 6.84 (d, J=7.2 Hz, 4H), 6.78˜6.72 (m, 10H), 6.67 (d, J=7.2 Hz, 6H), 6.47 (s, 2H), 2.10 (s, 6H); ¹³C NMR (CDCl₃, 100 MHz) δ 150.9, 149.5, 148.2, 147.1, 146.7, 145.3, 141.2, 141.1, 139.5, 139.4, 138.3, 136.1, 128.3, 127.3, 126.9, 126.5, 125.8, 123.6, 123.5, 123.4, 123.2, 122.8, 120.1, 120.0, 119.8, 119.7, 119.6, 119.0, 65.9, 64.4, 21.7; MS (m/z, FAB⁺) 992.3

In this embodiment, the mentioned conjugated compounds containing triarylamine structural elements are used in organic electroluminescence and/or phosphorescence devices, and more preferred, the compounds are used as hole transport material, hole injection material and/or as host material in organic electroluminescence and/or phosphorescence devices. Besides, the compounds can be used as hole transport material in solar cells.

### EXAMPLE 7

One chemical structure of the novel hole transporting or injecting materials is shown below, the chemical structure comprises aryl group Ar bounded to nitrogen atom, and aryl groups Ar¹ and Ar² on the 9-position of a fluorene unit. By selecting different Ar, Ar¹, and Ar² of the chemical structure, 7 analogues are provided as listed in Table 1, wherein compounds 4, 6, 7 are corresponding to example 3, 2, 1, respectively. The optical and electrochemical properties of the analogues are listed in Table 2. The HOMO levels of the mentioned analogues are about −4.9 eV, and are higher than those of NPB (about −5.09 eV). Such energy levels may provide a closer matching to the work function of ITO, when they are used as hole injection materials in OLEDs.

Furthermore, the thermal properties of the analogues in this example are determined by thermal gravimetric analysis (TGA) and differential scanning calorimetry (DSC). All analogues in this example show the glass transition temperatures from 182° C. to 279° C., which are much higher than conventional material NPB (T_{g} about 100° C.). Therefore, comparing with the OLEDs comprising conventional materials, the OLEDs comprising the analogues in this example can be operated at higher temperature, with less chance of crystallization, and with longer lifetime. In addition, all analogues in this example also show the higher degradation temperatures from 423° C. to 532° C. comparable to conventional material NPB.

TABLE 1

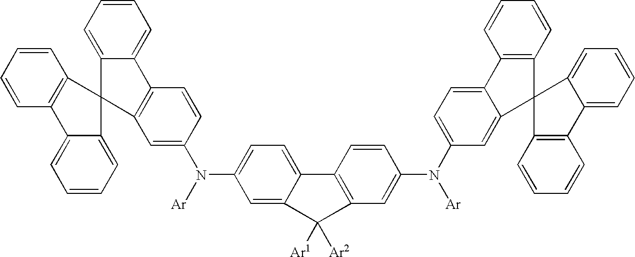

| Cpd | Ar | Ar¹ | Ar² | Cpd | Ar | Ar¹ | Ar² |
|---|---|---|---|---|---|---|---|
| 1 | 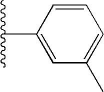 | p-tolyl | p-tolyl | 5 | 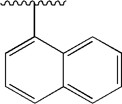 | p-tolyl | p-tolyl |
| 2 | 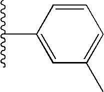 | p-tolyl | Phenyl | 6 | 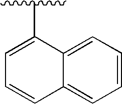 | p-tolyl | Phenyl |

| | | | | | | |
|---|---|---|---|---|---|---|
| 3 | 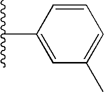 | Phenyl | phenyl | 7 | spirobifluorene | 2,2′-biphenyl |

| | | |
|---|---|---|
| 4 | 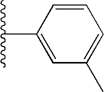 | 2,2′-biphenyl |

TABLE 2

| | | | Energy | CV | | | | | |
|---|---|---|---|---|---|---|---|---|---|
| | Abs.^{a} | Em.^{a} | Gap^{b} | oxidation | | HOMO^{c} | LUMO^{d} | | |
| Cpd | (nm) | (nm) | (eV) | (V) | | (eV) | (eV) | T_{g} (° C.) | T_{d} (° C.) |
| 1 | 393 | 420 | 2.93 | 0.64 | 0.98 | 4.96 | 2.03 | 186 | 445 |
| 2 | 390 | 424 | 2.89 | 0.62 | 0.96 | 4.94 | 2.05 | 183 | 445 |
| 3 | 384 | 436 | 2.92 | 0.62 | 0.96 | 4.94 | 2.02 | 182 | 495 |
| 4 | 382 | 426 | 2.92 | 0.63 | 0.96 | 4.95 | 2.03 | 190 | 423 |
| 5 | 380 | 452 | 2.92 | 0.64 | 1.01 | 4.96 | 2.04 | 209 | 532 |
| 6 | 380 | 453 | 2.93 | 0.64 | 1.01 | 4.96 | 2.03 | 207 | 531 |
| 7 | 381 | 431 | 2.85 | 0.61 | 0.94 | 4.93 | 2.08 | 279 | 513 |
| NPB | 344 | 465 | 3.02 | 0.75 | 1.03 | 5.09^{e} | 2.07^{e} | 100^{e} | 421^{e} |

^{a}Determined in Ethyl Acetate, spectrophotometric grade.
^{b}estimated from UV onset.
^{c}HOMO = 1^{st} oxidation − 0.48 + 4.8
^{d}LUMO = HOMO − Energy gap
^{e}Reported HOMO/LUMO = 5.12/2.12, reported T_{g}/T_{d} = 100° C./479° C., J. Mater. Chem., 2004, 14, 895-900

### EXAMPLE 8

The comparison of the optical and electrochemical properties of conventional material (NPB) and the mentioned compounds 4, 5, 6 is listed in Table 3. The compounds 4, 5, 6 respectively comprise one, two, four aromatic unit(s) as their central units in the general formula ABₘ. According to Table 3, the compounds, except for compound of Ex. 6, show HOMO levels higher than those of NPB. Furthermore, the thermal properties of the mentioned compounds are determined by DSC and TGA measurements, but DSC shows no evidence of the glass transition for compound of Ex. 5. As expected, most compounds in this example show higher T_{g}/T_{d} comparable to commercially used NPB, and among these compounds, T_{g}/T_{d} increase with increasing benzene numbers.

TABLE 3

| | | | Ener- | | | | | |
|---|---|---|---|---|---|---|---|---|
| | | | gy | CV | | | | |
| | Abs.^{a} | Em.^{a} | Gap^{b} | oxidation | HOMO^{c} | LUMO^{d} | T_{g} | T_{d} |
| Cpd | (nm) | (nm) | (eV) | (V) | (eV) | (eV) | (° C.) | (° C.) |
| Ex. 4 | 368 | 419 | 3.06 | 0.61, 0.88, | 4.95 | 1.89 | 178 | 473 |
| | | | | 1.16 | | | | |
| Ex. 5 | 356 | 406 | 3.20 | 0.69, 0.94 | 5.03 | 1.83 | N/A | 438 |
| Ex. 6 | 356 | 408 | 3.18 | 0.86 | 5.20 | 2.02 | 192 | 514 |
| NPB | 344 | 465 | 3.02 | 0.75, 1.03 | 5.09^{e} | 2.07^{e} | 100^{e} | 421^{e} |

^{a}Determined in CH₂Cl₂, spectrophotometric grade.
^{b}estimated from UV onset.
^{c}HOMO = 1^{st} oxidation − 0.46 + 4.8
^{d}LUMO = HOMO − Energy gap
^{e}Reported HOMO/LUMO = 5.12/2.12, reported T_{g}/T_{d} = 100° C./479° C., J. Mater. Chem., 2004, 14, 895-900

In a second embodiment of the present invention, a method for forming conjugated compounds containing triarylamine structural elements is disclosed. spiroarylamines (m moles) reacts with a multihalo-aryl compound (AXₘ) and a base in the presence of a palladium component, wherein m is a natural number from 2 to 4, X is halogen, and spiroarylamine has structure of the following: 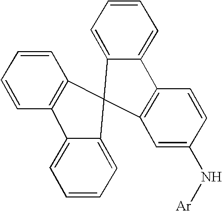
wherein A and Ar have been described in the first embodiment of the present invention. Referring to scheme 1, in a preferred example of this embodiment, m is 2, Ar¹ and Ar² have been described in the first embodiment, and AX₂ is 2,7-dibromo-9,9-diaryl-fluorene. 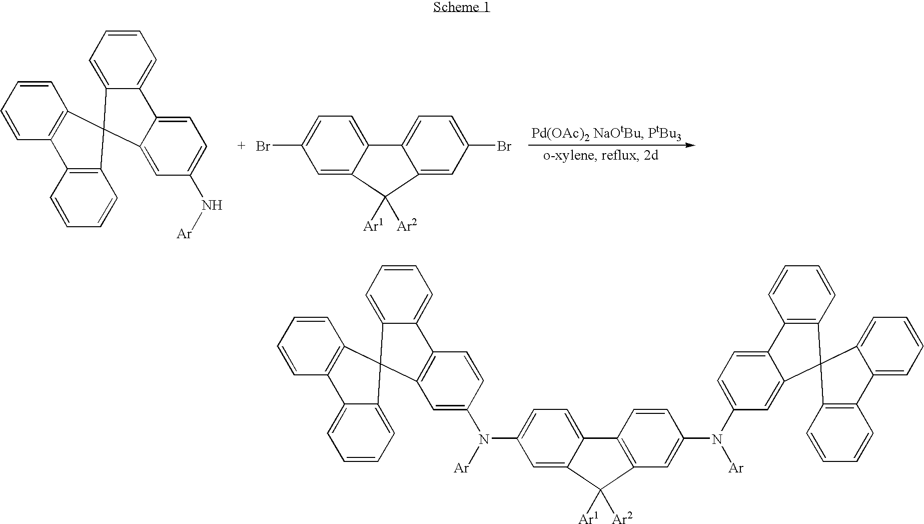
The detailed steps of the Scheme 1 are described as following. In a 250 mL, two-necked, round-bottomed flask was placed 2,7-dibromo-9,9-diaryl-fluorene (1 eq.), spiroarylamine (2.2 eq.), Pd(OAc)₂ (5 mol %) and Na(O^{t}Bu) (10 eq.). After degassed, o-xylene and P(^{t}Bu)₃ (20 mol %) were subsequently added, and the mixture was heated to reflux for 2 days. After completion of the reaction, ethyl acetate (EA) was added to the reaction mixture, and then the organic layer was separated, washed, and evaporated to get crude product. The crude product was purified by column chromatography on silica gel (EA/Hexane=1/7), so as to obtain light-yellow solid product.

In this embodiment, the method for forming the mentioned spiroarylamine comprises the steps of: 1. providing arylamine (H₂N—Ar) 2. providing halo-spiro compound 3. reacting arylamine (H₂N—Ar) with a halo-spiro compound and a base in the presence of a palladium component, as shown in Scheme 2. 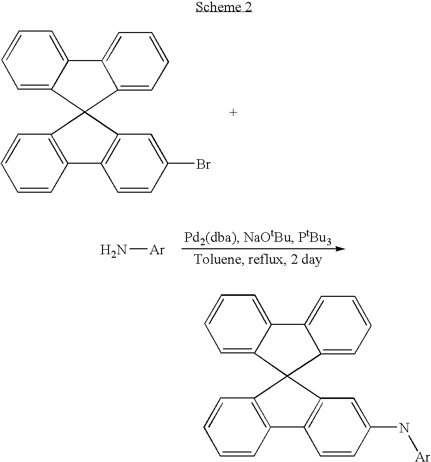
The detailed steps of the Scheme 2 are described as following. In a 500 mL, two-necked, round-bottomed flask was placed 2-bromo-9,9-diarylfluorene (40 mmol, 1 eq), arylamine (40 mmol, 1 eq), Pd₂(dba)₃ (1 mmol, 2.5 mol %), Na^{t}OBu (80 mmol, 2 eq.), P^{t}-Bu₃ (1.6 mmol, 4 mol %) and toluene (200 ml). The mixture was heated to reflux for 2 days. After completion of the reaction, crude product was purified by column chromatography on silica gel (Hexane/EA=4/1), so as to obtain final product (yield 80%).

### EXAMPLE 9

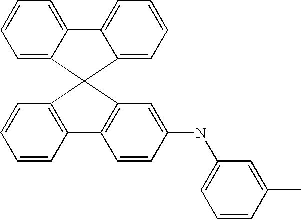

IR (CH₂Cl₂) ν 3054, 1635, 1422, 1265, 896, 744 cm⁻¹; ¹H NMR (acetone-d₆, 400 MHz) δ 7.84 (d, J=7.2 Hz, 2H), 7.72 (t, J=7.2 Hz, 2H), 7.28 (t, J=7.2 Hz, 2H), 7.23 (t, J=7.4 Hz, 1H), 7.15 (s, 1H), 7.09 (dd, J=8.2, 2.2 Hz, 1H), 7.05 (t, J=7.6 Hz, 2H), 6.91 (dd, J=7.8, 7.5 Hz, 2H), 6.70-6.67 (m, 2H), 6.63 (d, J=8.0 Hz, 2H), 6.51 (d, J=8.0 Hz, 2H), 6.24 (d, J=2.0 Hz, 3H), 2.15 (s, 1H); ¹³C NMR (acetone-d₆, 100 MHz) δ 150.4, 149.2, 147.9, 144.0, 143.2, 142.3, 141.7, 138.6, 133.8, 129.0, 127.9, 126.4, 123.9, 123.6, 121.3, 121.0, 120.3, 119.2, 118.1, 116.3, 114.9, 112.6, 112.5, 66.4; MS (m/z, FAB⁺) 421.2 (100%).

### EXAMPLE 10

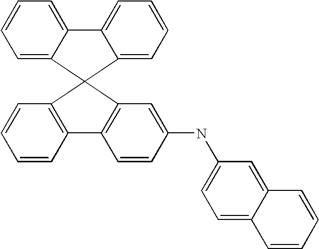

IR (CH₂Cl₂) ν 3442, 3054, 1634, 1266, 896, 728 cm⁻¹; ¹H NMR (acetone-d₆, 400 MHz) δ 8.05 (d, J=8.4 Hz, 1H), 7.92 (d, J=8.0 Hz, 1H), 7.84-7.80 (m, 3H), 7.49-7.25 (m, 9H), 7.18-7.12 (m, 3H), 7.05-7.01 (m, 1H), 6.75 (d, J=7.6 Hz, 2H), 6.59 (d, J=7.6 Hz, 1H), 6.39(d, J=2.4 Hz, 1H); ¹³C NMR (acetone-d₆, 100 MHz) δ 150.5, 149.3, 148.1, 145.5, 142.4, 141.2, 139.3, 135.0, 133.9, 128.4, 128.1, 128.0, 127.7, 126.5, 126.2, 126.1, 125.4, 124.0, 123.7, 122.6, 122.4, 121.1, 120.4, 119.3, 116.6, 115.0, 112.9, 66.5; MS (m/z, FAB⁺) 457.2 (100%).

### EXAMPLE 11

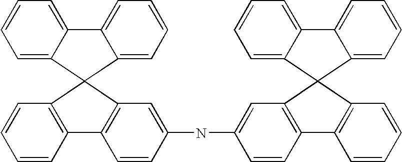

¹H NMR (acetone-d₆, 400 MHz) δ 7.14 (d, J=8.0 Hz, 2H), 7.01 (d, J=7.2 Hz, 1H), 6.93 (d, J=7.2 Hz, 1H), 6.61˜6.51 (m, 4H), 6.33 (t, J=7.2 Hz, 2H), 6.27˜6.20 (m, 4H), 5.86 (d, J=7.2 Hz, 2H), 5.76 (d, J=8.0 Hz, 1H), 5.46 (d, J=2.0 Hz, 1H); MS (m/z, FAB⁺) 645.3 (100%).

Synthesis of Arylamine (H₂N—Ar)

arylamines with simple structures can be perchased, such as: 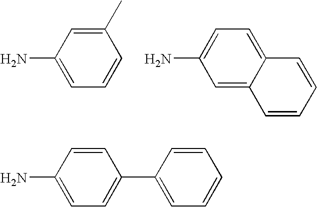
arylamines with complicated structures, such as: spiroamine, can be synthesized as shown in Scheme 3: 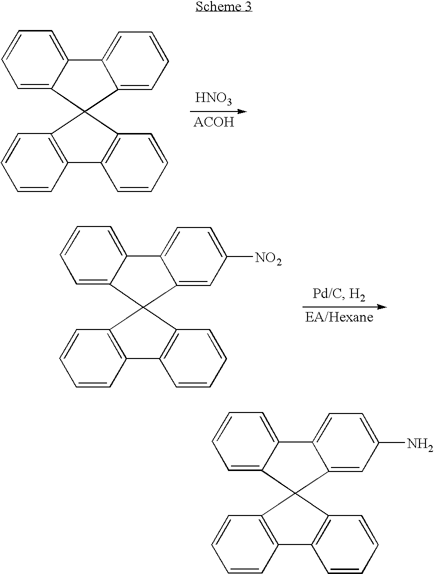
The detailed steps are described as following. Into a 250 mL, two-necked, round-bottomed flask was added a solution of spirobifluorene (1.0 g, 3.16 mmol) in acetic acid (50 mL). the solution was stirred at 90° C. to effect their complete dissolution, and then nitric acid (6 mL) was added drop by drop. After completion of the reaction (30 mins) as monitored by TLC, the reaction mixture with products (nitro group on 2 or 2′ position) was cooled to room temperature and quenched with water, so that yellow solid was precipitated. Yellow solid was then washed, filtered, and purified by column chromatography to form light-yellow solid 2-nitrospirobifluorene (0.70 g, 88%). Afterwards, in a 500 mL, two-necked, round-bottomed flask was placed 2-nitrospirobifluorene (6.70 g, 19.18 mmol), Pd/C (10%, 0.41 g, 3.84 mmol) and EA/ethanol(120/30 mL). After degassed, hydrogen was purged. The operation of degassing and hydrogen purging was repeated for about 10 times to fill the flask with hydrogen. The flask was further equipped with a hydrogen container for sufficient hydrogen supply. After completion of the reaction (at room temperature for 2 days), the reaction mixture was filtered and evaporated to form light-purple solid (4.50 g, 73%).
Synthesis of Multihalo-aryl Compound

multihalo-aryl compounds with simple structures can be perchased, such as: 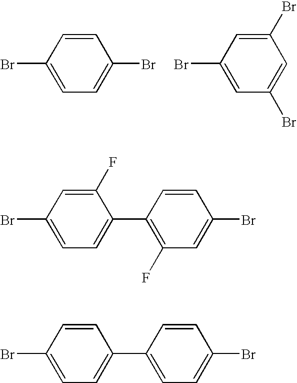
multihalo-aryl compounds with complicated structures, can be synthesized as shown in the following examples:

### EXAMPLE 12

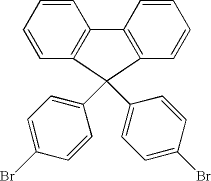

Into a 250 mL, two-necked, round-bottomed flask was added a solution of 9,9-bis(4-aminophenyl)fluorine (5 g) in H₂SO₄(aq.) (50 mL). The solution was then stirred for 10 minutes. Before complete dissolution, the flask with solution was ice bathed to lower the temperature of the solution to below 10° C. Next, NaNO₂ (2.14 g) was dissolved in 20 ml water, and then added to the cooled solution drop by drop. The mixture was stirred at temperature below 10° C. for 30 minutes. After stirred, the mixture was mixed with a solution of CuBr (20.5 g) in 43% HBr (200 ml) with temperature below 10° C. The mixture was heated to 50° C. and stirred for 3 hours, then cooled to room temperature. Afterwards, ethyl acetate (3×50 ml) was added to the reaction mixture, and then the organic layer was separated and washed by 3 M HCl. The organic layer was further separated, dehydrated by MgSO₄, and vacuum concentrated. Finally, crude product was purified by petroleum ether to get end product 5.7 g (84%).

### EXAMPLE 13

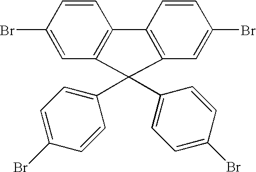

To a 100 mL, three-necked, flask was added a solution of 9,9-bis(4-bromophenyl)fluorine (5.7 g) in CH₂Cl₂ (30 mL). The solution was heated to reflux, and a solution of bromide (5.7 g) dissolved in 10 ml CH₂Cl₂ was added into the refluxing solution drop by drop. The mixture was stirred overnight. After completion of the reaction, the reaction mixture was washed by 80 ml water for 2 times and saturated K₂CO₃ (aq.) for 1 time. The organic layer was separated, dehydrated by MgSO₄, and vacuum concentrated to obtain product 4.7 g (62%).

### EXMPLE 14

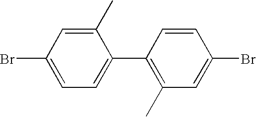

To a 250 mL flask was added a solution of 2,2′-dimethylbenzidine Hydrochloride (50 g) in HCl (aq.) (100 mL) The solution was stirred for 10 minutes, then 100 g of ice was added. the flask with solution and ice was ice bathed to lower the temperature of the solution to below 10° C. Next, NaNO₂ (24 g) was dissolved in 30 ml water, and then added to the cooled solution drop by drop. The mixture was mixed with a solution of CuBr (proper weight) in water (500 ml) with temperature below 10° C., and subsequently stirred overnight. Afterwards, ethyl acetate (2×500 ml) was added to the reaction mixture, and then the organic layer was separated and washed by NaOH(aq.). The organic layer was further separated, dehydrated by MgSO₄, and vacuum concentrated. Finally, crude product was purified by column chromatography to obtain wanted product 13 g.

### EXAMPLE 15

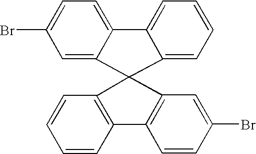

To a 100 mL, three-necked, flask was added a solution of spirobifluorene (10 g) in CH₂Cl₂ (100 mL). The solution was heated to reflux, and a solution of bromide (5 g) dissolved in 20 ml CH₂Cl₂ was added into the refluxing solution drop by drop. The mixture was then stirred overnight. After completion of the reaction, the reaction mixture was washed by 80 ml water for 2 times and saturated K₂CO₃(aq.) for 1 time. The organic layer was separated, dehydrated by MgSO₄, and vacuum concentrated to form crude product. Finally, crude product was purified by column chromatography to obtain wanted product 3.7 g (24%).

In a third embodiment of the present invention, an OLED comprising a multilayer structure for producing electroluminescence is provided. For realizing practical full-color displays, red-, green-, and blue-emitters with sufficiently high luminous efficiencies and color purity are required. Two common methods of tuning the color of an OLED are a) choosing an emission material with the appropriate intrinsic emission characteristics or b) incorporating in a host transport material guest dopants with the appropriate emission characteristics. Introducing dopants in organic molecular films facilitates the control of a number of device properties such as electroluminescence (EL) Quantum efficiency, thermal stability, durability, and carrier injection and transport.

Guest emitter is usually doped in host by co-evaporation or dispersion process, and receives energy from host in the way of energy transfer or carrier trap, so as to result in generating varying colors and enhancing the EL efficiency of OLEDs. On the other hand, upon electron and hole recombination, spin statistics limits the maximum production efficiency of singlet and triplet excitons to 25% and 75%, respectively. Hence, the incorporation of phosphor molecules in the emitting layer provides an additional radiative channel through triplet emission. The developing studies show most phosphorescence based emitters containing organic-metal complexes. The central unit of organic-metal complex is transient metal, such as: Os, Ir, Pt, Eu, and Ru, and the ligands are heterocycles containing nitrogen atom.

According to this embodiment, the mentioned multilayer structure comprises: a substrate; an anode layer; a hole transporting layer comprising a compound of a general formula ABₘ described in the first embodiment; an electron transporting layer; and a cathode layer. Additionally, a hole injecting layer can be located between the anode and the hole transporting layer. Furthermore, there are three examples about the configurations of emitting layer: 1. the hole transporting layer is emitting layer 2. the electron transporting layer is emitting layer 3. the emitting layer is located between the hole transporting layer and the electron transporting layer.

In a fourth embodiment of the present invention, a OLED comprising a multilayer structure for producing electroluminescence is provided, wherein the multilayer structure comprises: a substrate; an anode layer; a hole injecting layer comprising a compound of a general formula ABₘ described in the first embodiment; a hole transporting layer; an electron transporting layer; and a cathode layer. Moreover, there are three examples about the configurations of emitting layer: 1. the hole transporting layer is emitting layer 2. the electron transporting layer is emitting layer 3. the emitting layer is located between the hole transporting layer and the electron transporting layer.

General Method of Producing OLEDs

ITO-coated glasses with 15 Ω□⁻¹ and 1500 μm in thickness are provided (purchased from Sanyo vacuum, hereinafter ITO substrate) and cleaned in a number of cleaning steps in an ultrasonic bath (e.g. detergent, deionized water). Before vapor deposition of the organic layers, cleaned ITO substrates are further treated by UV and ozone.

The organic layers are applied onto the ITO substrate in order by vapor deposition in a high-vacuum unit (10⁻⁶ Torr), such as: resistively heated quartz boats. The thickness of the respective layer and the vapor deposition rate (0.1˜0.3 nm/sec) are precisely monitored or set with the aid of a quartz-crystal monitor.

It is also possible, as described above, for individual layers to consist of more than one compound, i.e. in general a host material doped with a guest material. This is achieved by covaporization from two or more sources.

Tris-(8-hydroxyquinoline) aluminum (Alq₃) is most widely used as the electron transport/light emitting layer in OLEDs for its high thermal stability and good film forming property.

A typical OLED consists of low work function metals, such as Al, Mg, Ca, Li and K, as the cathode by thermal evaporation, and the low work function metals can help electrons injecting the electron transporting layer from cathode. In addition, for reducing the electron injection barrier and improving the OLED performance, a thin-film electron injecting layer is introduced between the cathode and the electron transporting layer. Conventional materials of electron injecting layer are metal halide or metal oxide with low work function, such as: LiF, MgO, or Li₂O.

On the other hand, after the OLEDs are fabricated, EL spectra and CIE coordination are measured by using a PR650 spectra scan spectrometer. Furthermore, the current/voltage, luminescence/voltage and yield/voltage characteristics are taken with a Keithley 2400 programmable voltage-current source. The above-mentioned apparatuses are operated at room temperature (about 20° C.) and under atmospheric pressure.

### EXAMPLE 16

Using a procedure analogous to the abovementioned general method, four green-emitting OLEDs having the following structure was produced:
Device I-a: ITO/NPB (50 nm)/Alq₃(50 nm)/LiF(0.5 nm)/Al(150 nm)
Device I-b: ITO/Compound of example 1 (60 nm)/Alq₃(50 nm)/LiF(0.5 nm)/Al(150 nm)
Device I-c: ITO/Compound of example 2 (50 nm)/Alq₃(50 nm)/LiF(0.5 nm)/Al(150 nm)
Device I-d: ITO/Compound of example 3 (60 nm)/Alq₃(50 nm)/LiF(0.5 nm)/Al(150 nm)

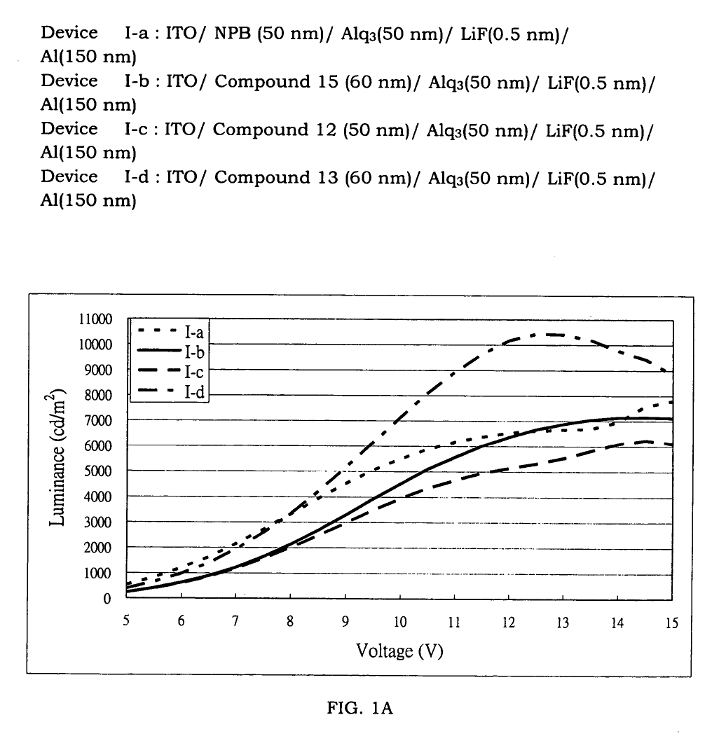

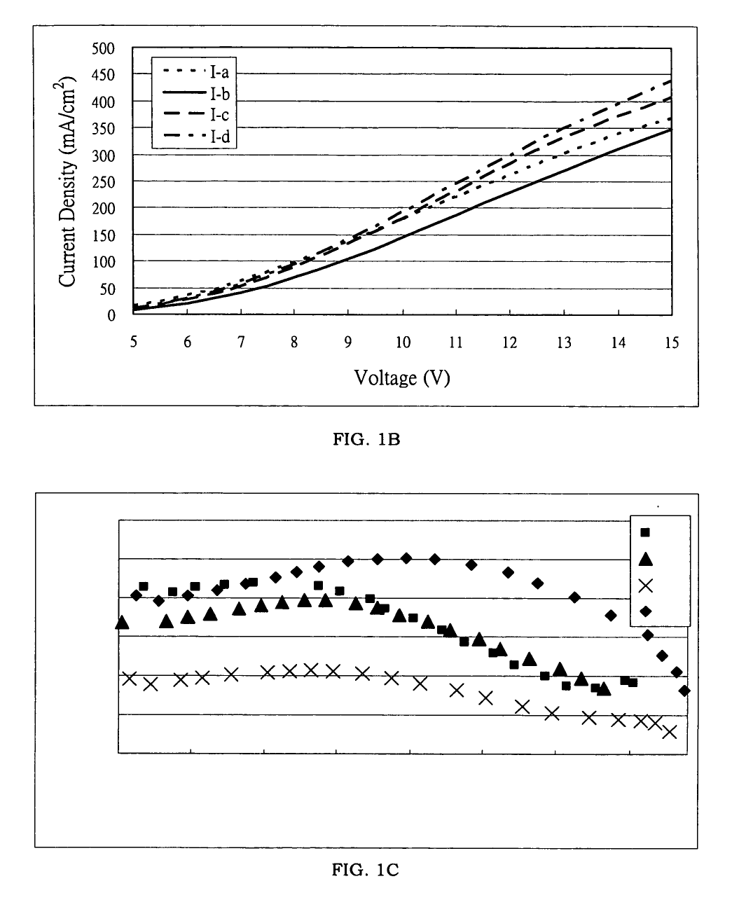

The hole transporting material of device I-a, I-b, I-c, and I-d are NPB, compound of example 1, compound of example 2, compound of example 3, respectively. Referring to FIG. 1A, Luminance-Voltage characteristics of device I-a, I-b, and I-c show the same trend that the brightness is increased with increasing driving voltage. The maximum brightness of device I-a is about 8000 cd/m² at a driving voltage of 15 V. In a comparison, the maximum brightness of device I-d is about 10500 cd/m² at a driving voltage ranges from 12 V to 13 V.

It is interesting to notice that, while there is about 30% increase in the maximum brightness when comparing device I-d versus NPB-based device I-a, at the same time the driving voltage of device I-d is lower than that of device I-a. Besides, when driving voltage ranges from 10 V to 15 V, brightness of device I-d are larger than 8000 cd/m², which means device I-d provided in this invention can be operated under varied driving voltages with matching requirements for high brightness.

FIG. 1B shows voltage-current density characteristics of the devices. All characteristics show the same trend, and current density is increased with increasing driving voltage. When driving voltages are the same, current density of device I-d is larger than that of device I-a; in another word, when current densities are the same, driving voltage of device I-d is smaller than that of device I-a. Therefore, device I-d shows higher circuit efficiency than NPB-based device I-a.

Referring to FIG. 1C, when current density increased to 300 mA/cm², EL efficiency of device I-a, I-b, and I-c are dramatically decreased to 2 cd/A, but only device I-d still with EL efficiency above 3 cd/A. This shows device I-d provided in this invention is able to maintain high EL efficiency with matching requirements for high brightness (brightness larger than 8000 cd/m², driving voltage larger than 10V, and current density larger than 200 mA/cm²). In contrast, NPB-based device I-a exhibits EL efficiency lower than 2 cd/A with matching requirements for high brightness (brightness larger than 7000 cd/m², driving voltage larger than 14V, and current density larger than 200 mA/cm²). Therefore, for NPB-based device I-a, high brightness and high EL efficiency can not be achieved at the same time.

### EXAMPLE 17

Using a procedure analogous to the abovementioned general method, four blue-emitting OLEDs having the following structure was produced:

Device II-a:

ITO/2T-NATA(10 nm)/NPB (35 nm)/BCzVBi 5% doped DPVBi(35 nm)/Alq₃(15 nm)/LiF(0.5 nm)/Al(150 nm)
   Device II-b:
ITO/2T-NATA(10 nm)/Compound of example 4 (35 nm)/BCzVBi 5% doped DPVBi(35 nm)/Alq₃(15 nm)/LiF(0.5 nm)/Al(150 nm)
   Device II-c:
ITO/2T-NATA(10 nm)/Compound of example 5(35 nm)/BCzVBi 5% doped DPVBi(35 nm)/Alq₃(15 nm)/LiF(0.5 nm)/Al(150 nm)
   Device II-d:
ITO/2T-NATA(10 nm)/Compound of example 6 (35 nm)/BCzVBi 5% doped DPVBi(35 nm)/Alq₃(15 nm)/LiF(0.5 nm)/Al(150 nm)

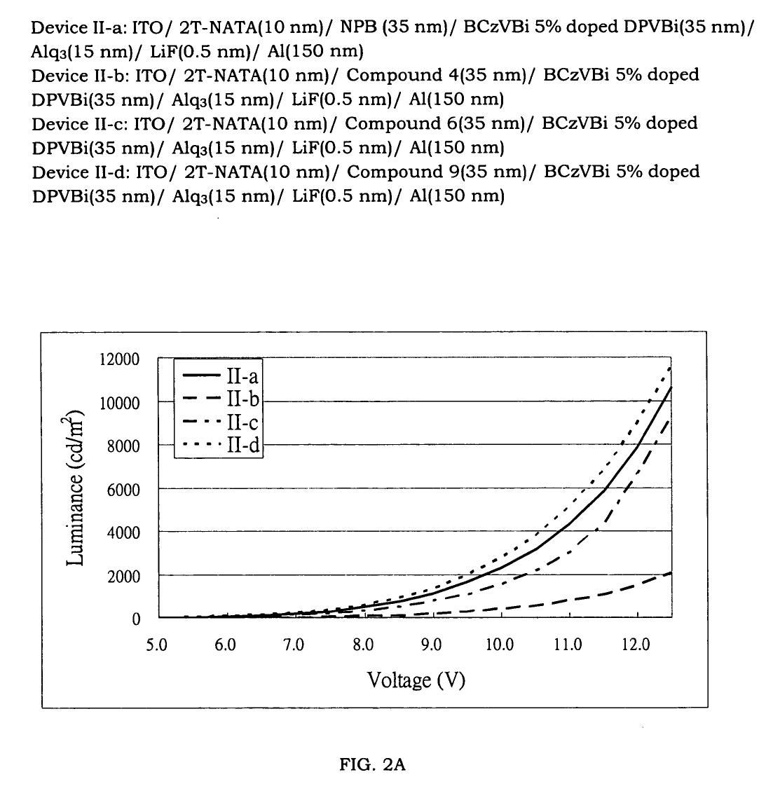

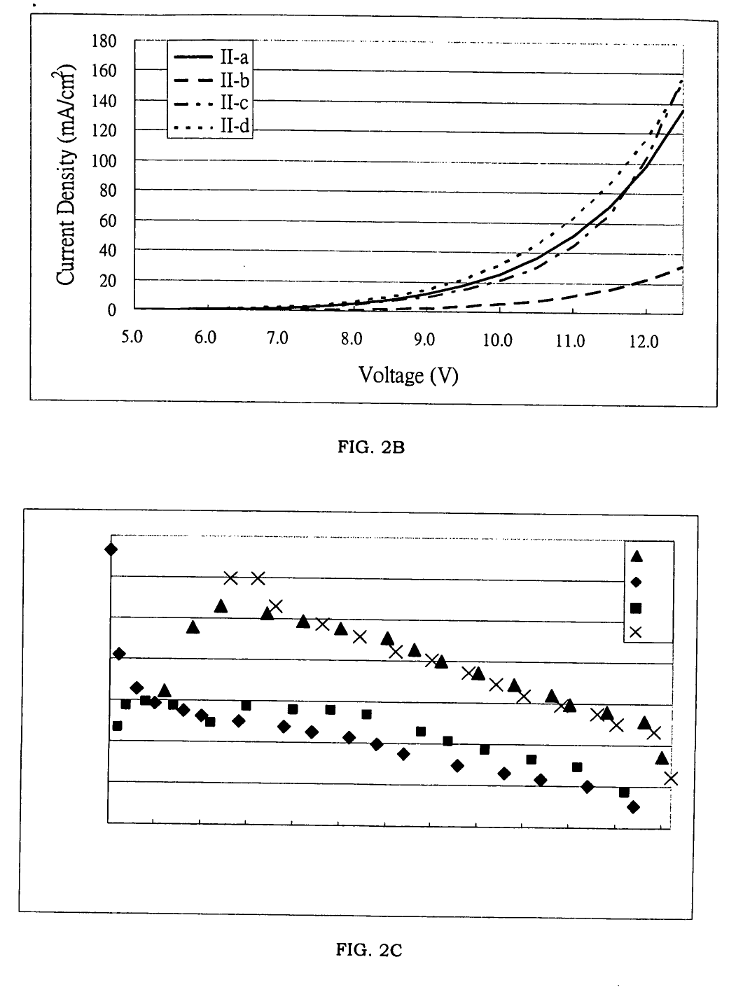

The hole transporting material of device II-a, II-b, II-c, and II-d are NPB, compound of example 4, compound of example 5, compound of example 6, respectively. Referring to FIG. 2A, when driving voltage is larger than 10V, Luminance-Voltage characteristics of device II-a, II-c, and II-d show the same trend, and brightness is increased with increasing driving voltage. Device II-a, II-c, and II-d exhibit larger brightness than that of device II-b. Furthermore, the brightness of device II-d is about 9000 cd/m² at a driving voltage of 12V, while the brightness of NPB-based device II-a is 8000 cd/m². Therefore, device II-d provided in this invention exhibits higher brightness than that of NPB-based device under the same driving voltage.

FIG. 2B shows voltage-current density characteristics of the devices. All characteristics show the same trend, and current density is increased with increasing driving voltage. When driving voltages are the same, current density of device II-a, II-c, and II-d are larger than that of device II-b, wherein device II-d exhibits the highest circuit efficiency. When driving voltage is smaller than 11.5V, device II-a exhibits higher circuit efficiency than device II-c; but when driving voltage is larger than 11.5V, device II-c exhibits higher circuit efficiency than device II-a. Therefore, device II-d provided in this invention shows higher circuit efficiency than NPB-based device I-a, and so does device II-c under high driving voltage.

FIG. 2C shows EL efficiency-current density characteristics of the devices, wherein characteristic of device II-a is similar to that of device II-d, and characteristic of device II-b is similar to that of device II-c. EL efficiencies of device II-a and II-d are high than those of device II-b and II-c. It is noteworthy that device II-d provided in this invention shows higher EL efficiency than NPB-based device II-a in the low current density range of 1-4 mA/cm², and in other current density range, characteristics of device II-a and II-d are very close.

In the above preferred embodiments, the present invention make spiroarylamines react with halo aryl compounds and a base in the presence of a palladium component, so as to form the mentioned conjugated compounds containing triarylamine structural elements. Moreover, the present invention provides a multilayered OLED with high brightness, high efficiency, and high thermal stability, wherein the OLED comprises the mentioned conjugated compounds containing triarylamine structural elements as hole transporting material or hole injecting material. Comparing with conventional material NPB, the glass transition temperature (T_{g}) and thermal degradation temperature (T_{d}) are 279° C. and 532° C. respectively. The thermal property of conjugated compounds in this invention is obviously better than that of NPB. Therefore, this present invention does have the economic advantages for industrial applications.

To sum up, the present invention discloses conjugated compounds containing triarylamine structural elements with a general formula ABₘ, wherein A is aryl moiety with or without hetero atom(s), m is integers from 2 to 4, and B is as following: 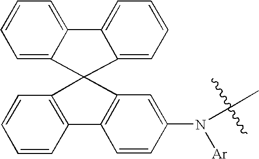
wherein Ar is aryl moiety. Moreover, the conjugated compounds of the invention are used as hole transport material, hole injection material and/or as host material in organic electroluminescence devices and/or phosphorescence devices, and as hole transport material in solar cells.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A conjugated compound containing triarylamine structural elements with a general formula ABₘ, wherein A is aryl moiety with or without hetero atom(s), m is integers from 2 to 4, and B is as following: 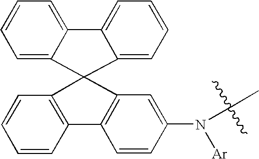
wherein Ar is aryl moiety.

2. The compound as claimed in claim 1, wherein A is selected from the group consisting of:

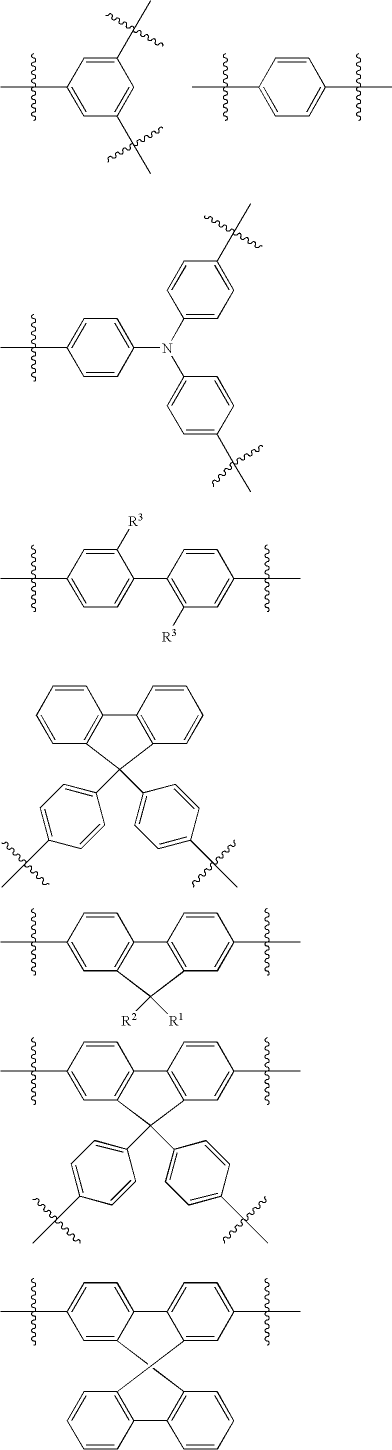

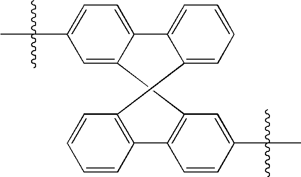

3. The compound as claimed in claim 2, wherein R¹ and R² are identical or different, and R¹ and R² are independently selected from the group consisting of: linear alkyl, branched alkyl, cyclic alkyl, aryl moiety, hetero cycle, multiple fused ring, multiple fused ring with hetero atom(s), and alkyl with at least one substitute of alkene or alkyne or halogen or alkoxy or siloxane or ketone or alcohol or thioether or carbamates, or amine.

4. The compound as claimed in claim 2, wherein R³ is selected from the group consisting of: hydrogen, halogen, linear alkyl, branched alkyl, cyclic alkyl, aryl moiety, hetero cycle, multiple fused ring, multiple fused ring with hetero atom(s), and alkyl with at least one substitute of alkene or alkyne or halogen or alkoxy or siloxane or ketone or alcohol or thioether or carbamates, or amine.

5. The compound as claimed in claim 1, wherein Ar is selected from the group consisting of:

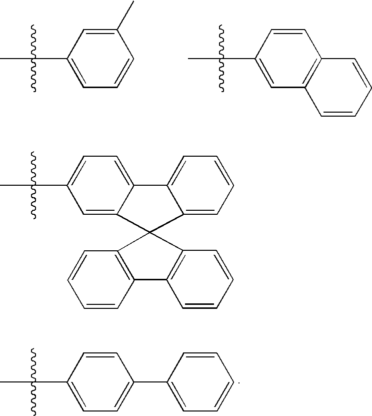

6. The compound as claimed in claim 1, wherein said compound is used in organic electroluminescence and/or phosphorescence devices.

7. The compound as claimed in claim 1, wherein said compound is used as hole transport material, hole injection material and/or as host material in organic electroluminescence and/or phosphorescence devices.

8. The compound as claimed in claim 1, wherein said compound is used as hole transport material in solar cells.

9. An organic light emitting device comprising a multilayer structure for producing electroluminescence, wherein the multilayer structure comprises:
a substrate;
an anode layer;
a hole transporting layer comprising a compound of a general formula ABₘ, wherein A is aryl moiety with or without hetero atom(s), m is integers from 2 to 4, and B is as following: 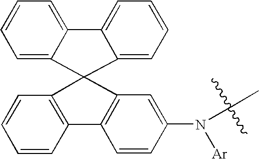
wherein Ar is aryl moiety;
an electron transporting layer; and
a cathode layer.

10. The organic light emitting device as claimed in claim 9, wherein A is selected from the group consisting of:

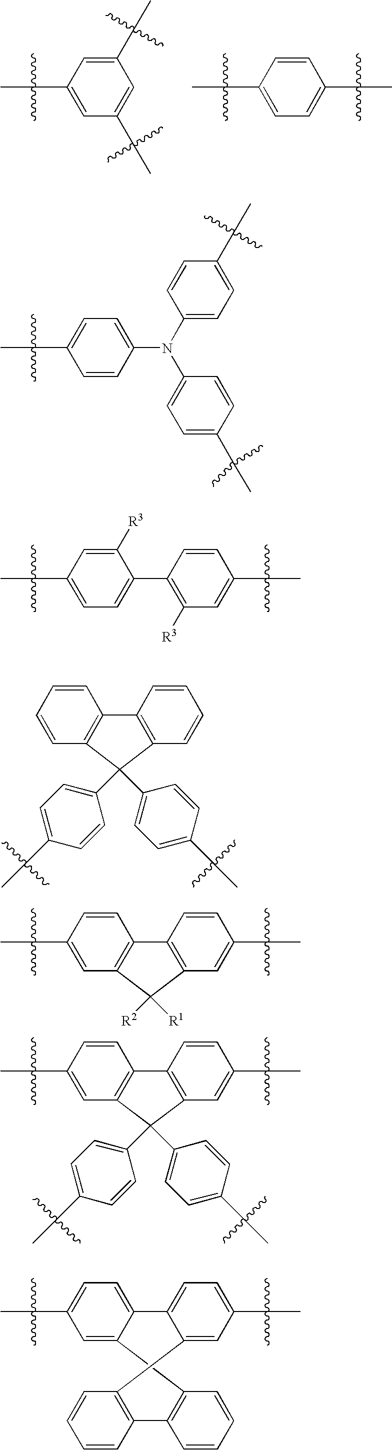

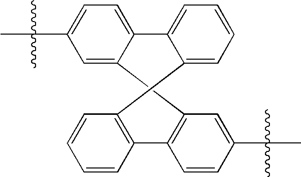

11. The organic light emitting device as claimed in claim 10, wherein R¹ and R² are identical or different, and R¹ and R² are independently selected from the group consisting of: linear alkyl, branched alkyl, cyclic alkyl, aryl moiety, hetero cycle, multiple fused ring, multiple fused ring with hetero atom(s), and alkyl with at least one substitute of alkene or alkyne or halogen or alkoxy or siloxane or ketone or alcohol or thioether or carbamates, or amine.

12. The organic light emitting device as claimed in claim 10, wherein R³ is selected from the group consisting of: hydrogen, halogen, linear alkyl, branched alky, cyclic alkyl, aryl moiety, hetero cycle, multiple fused ring, multiple fused ring with hetero atom(s), and alkyl with at least one substitute of alkene or alkyne or halogen or alkoxy or siloxane or ketone or alcohol or thioether or carbamates, or amine.

13. The organic light emitting device as claimed in claim 9, wherein Ar is selected from the group consisting of:

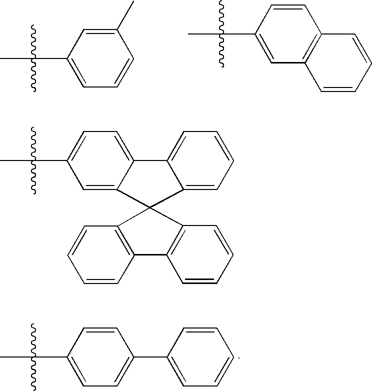

14. An organic light emitting device comprising a multilayer structure for producing electroluminescence, wherein the multilayer structure comprises:
a substrate;
an anode layer;
a hole injecting layer comprising a compound of a general formula ABₘ, wherein A is aryl moiety with or without hetero atom(s), m is integers from 2 to 4, and B is as following: 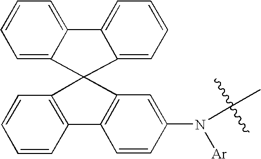
wherein Ar is aryl moiety;
a hole transporting layer;
an electron transporting layer; and
a cathode layer.

15. The organic light emitting device as claimed in claim 14, wherein A is selected from the group consisting of:

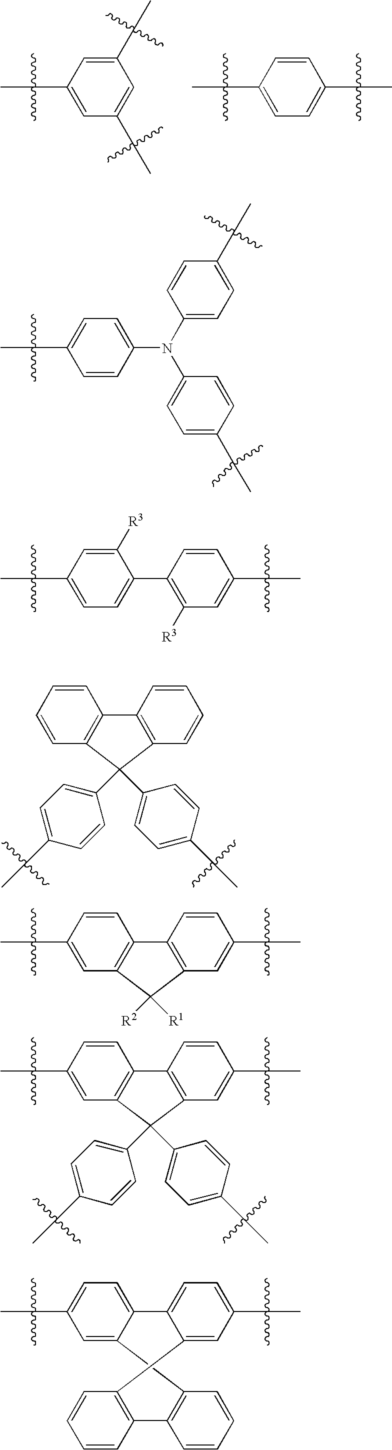

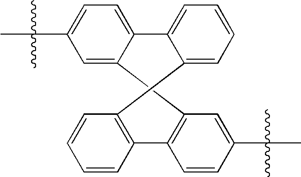

16. The organic light emitting device as claimed in claim 15, wherein R¹ and R² are identical or different, and R¹ and R² are independently selected from the group consisting of: linear alkyl, branched alkyl, cyclic alkyl, aryl moiety, hetero cycle, multiple fused ring, multiple fused ring with hetero atom(s), and alkyl with at least one substitute of alkene or alkyne or halogen or alkoxy or siloxane or ketone or alcohol or thioether or carbamates, or amine.

17. The organic light emitting device as claimed in claim 15, wherein R³ is selected from the group consisting of: hydrogen, halogen, linear alkyl, branched alkyl, cyclic alkyl, aryl moiety, hetero cycle, multiple fused ring, multiple fused ring with hetero atom(s), and alkyl with at least one substitute of alkene or alkyne or halogen or alkoxy or siloxane or ketone or alcohol or thioether or carbamates, or amine.

18. The organic light emitting device as claimed in claim 14, wherein Ar is selected from the group consisting of: [chemical structure]
